(19) United States
(12) Patent Application Publication
(10) Pub. No.: US 2024/0024599 A1
(43) Pub. Date: Jan. 25, 2024

(54) Artificial Intelligence Oxygen Controller, Blood Oxygen Measurement Device therewith and Artificial Intelligence Oxygen Control and System therewith

(71) Applicant: Kuo-Yuan CHANG, New Taipei City (TW)

(72) Inventor: Kuo-Yuan CHANG, New Taipei City (TW)

(21) Appl. No.: 17/869,415

(22) Filed: Jul. 20, 2022

(51) Int. Cl. A61M 16/00; A61M 16/10

(52) U.S. Cl. CPC A61M 16/022; A61M 16/1005; A61M 2205/3334; A61M 2205/3553; A61M 2230/20; A61M 2202/0208; A61M 2202/0007

(57) ABSTRACT

An artificial intelligence oxygen controller includes an oxygen input receiving a first flow rate of gas containing high-concentration oxygen, an oxygen output outputting a second flow rate of gas containing high-concentration oxygen, and a gas regulation-output module receiving a blood oxygen level and comparing the blood oxygen level with a blood oxygen threshold range, where an amount of second flow rate of gas containing high-concentration oxygen from the first flow rate of gas containing high-concentration oxygen is determined by a comparison result. There are an artificial intelligence oxygen control system therewith, a blood oxygen measurement device therewith, and an artificial intelligence oxygen supply device therewith.

### 1. FIELD OF THE INVENTION

The present invention relates to an oxygen supply and control technology, particularly to an adaptive oxygen supply system with artificial intelligent connecting blood oxygen measure system.

### 2. DESCRIPTION OF THE PRIOR ART

Homecare is becoming a trend now. High-concentration oxygen supply is an indispensable function for homecare. In the current high-concentration oxygen supply technology, an oxygen supplier or generator is directly assembled to a tube and a respiration mask for supplying oxygen to the user. Alternatively, a switch is disposed on the oxygen supplier or generator; a third party observes the condition of the user and determines whether to operate the switch to supply oxygen to the user.

However, the physiological status of the user may change any time. Persistently supplying high-concentration oxygen to the user who has got better may be unfavorable to the user. Therefore, such a situation needs improving.

### SUMMARY OF THE INVENTION

One objective of the present invention is to provide an artificial intelligence oxygen controller and an integrated device and system therewith, wherein the instant blood oxygen level is used as a basis for controlling the amount of oxygen supplied to a user, whereby oxygen may be appropriately supplied to the user according to his own instant blood oxygen level.

Another objective of the present invention is to provide an artificial intelligence oxygen controller and an integrated device and system therewith, wherein the result of the comparison between the instant blood oxygen level and the blood oxygen threshold range is used to control the regulation valve and determine whether the high-concentration oxygen from an oxygen supplier is partially or completely provided, whereby oxygen may be adaptively supplied to the user.

In order to achieve the abovementioned objectives, an artificial intelligence oxygen controller includes an oxygen input receiving a first flow rate of gas containing high-concentration oxygen, an oxygen output outputting a second flow rate of gas containing high-concentration oxygen, and a gas regulation-output module receiving a blood oxygen level and comparing the blood oxygen level with a blood oxygen threshold range, where an amount of second flow rate of gas containing high-concentration oxygen from the first flow rate of gas containing high-concentration oxygen is determined by a comparison result.

Accordingly, an artificial intelligence oxygen control system including the artificial intelligence oxygen controller includes a blood oxygen measurement device measuring a physiological response signal of a user, processing the physiological response signal to acquire the blood oxygen level and transmitting the blood oxygen level to the artificial intelligence oxygen controller; and an oxygen supply device providing the first flow rate of gas containing high-concentration oxygen to the artificial intelligence oxygen controller, wherein a concentration of oxygen in the first flow rate of gas containing high-concentration oxygen is more than 60%.

Accordingly, a blood oxygen measurement device including the artificial intelligence oxygen controller includes a physiological signal interface outputting a detection signal and receiving a physiological response signal of the user, wherein the detection signal interferes with the user to generate the physiological response signal of the user; a signal processing-analyzing module connected with the physiological signal interface and processing the physiological response signal to acquire a measurement result including the blood oxygen level; a transmission module connected with signal processing-analyzing module, transmitting the measurement result to an external device for presentation in a wireless or wired way; and a display module connected with the signal processing-analyzing module for displaying the measurement result.

Accordingly, an artificial intelligence oxygen supply device including the artificial intelligence oxygen controller includes an oxygen supplier providing the first flow rate of gas containing high-concentration oxygen to the artificial intelligence oxygen controller, wherein a concentration of oxygen in the first flow rate of gas containing high-concentration oxygen is more than 60%.

### BRIEF DESCRIPTION OF THE DRAWINGS

FIG. 1 is a diagram schematically showing an artificial intelligence oxygen control system according to a first embodiment of the present invention.

FIG. 2 is a diagram schematically showing an artificial intelligence oxygen control system according to a second embodiment of the present invention.

FIG. 3 is a diagram schematically showing an artificial intelligence oxygen control system according to a third embodiment of the present invention.

FIG. 4 is a diagram schematically showing an artificial intelligence oxygen control system according to a fourth embodiment of the present invention.

FIG. 5 is a diagram schematically showing an artificial intelligence oxygen control system according to a fifth embodiment of the present invention.

### DESCRIPTION OF THE PREFERRED EMBODIMENT

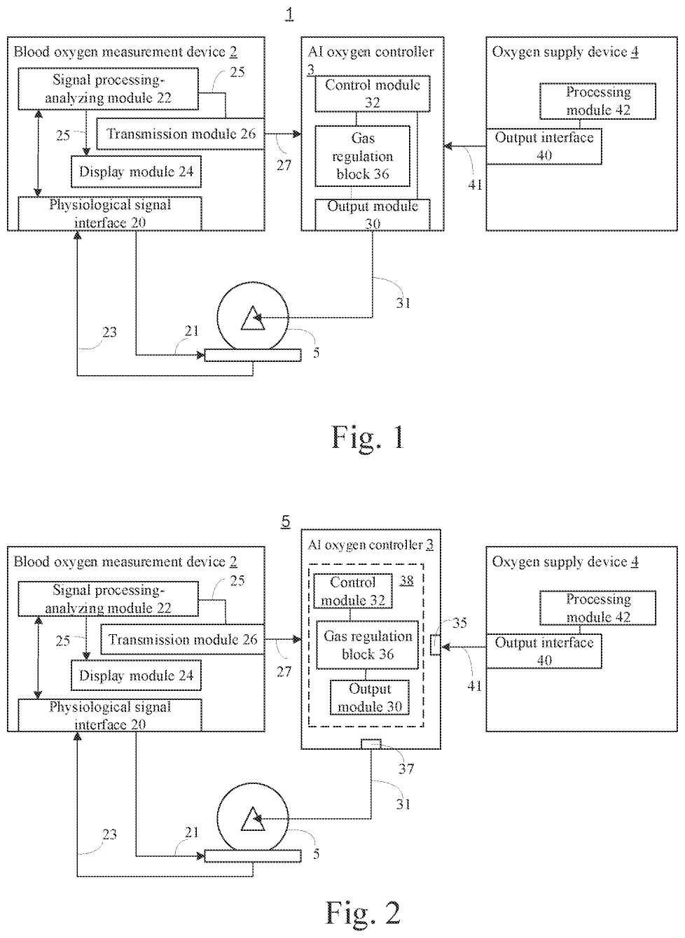

FIG. 1 is a diagram schematically showing an artificial intelligence oxygen control system according to a first embodiment of the present invention. The artificial intelligence oxygen control system 1 shown in FIG. 1 includes a blood oxygen measurement device 2, an artificial intelligence oxygen controller 3, and an oxygen supply device 4. The blood oxygen measurement device 2 measures one or more physiological signals of a user 5. The oxygen supply device 4 generates and supplies a gas containing high-concentration oxygen. The artificial intelligence oxygen controller 3 is connected with the blood oxygen measurement device 2 and the oxygen supply device 4. The artificial intelligence oxygen controller 3 operates to supply a gas containing oxygen at a specified flow of high-concentration oxygen to the user 5 according to the blood oxygen level measured by the blood oxygen measurement device 2.

Refer to FIG. 1 again. The blood oxygen measurement device 2 includes a physiological signal interface 20 to output a detection signal 21 to act on the user 5. A physiological response signal 23 of the user 5 is fed back to the blood oxygen measurement device 2 through the physiological signal interface 20 for further processing. The blood oxygen measurement device 2 also includes a signal processing-analyzing module 22, a display module 24 and a transmission module 26. The signal processing-analyzing module 22 is connected with the physiological signal interface 20 and the display module 24. The physiological response signal 23 is transmitted to the signal processing-analyzing module 22 through the physiological signal interface 20. The signal processing-analyzing module 22 analyzes the physiological response signal 23 and transmits a measurement result 25 to the display module 24, whereby the user 5 can view and learn the measurement result 25. In the first embodiment, the blood oxygen measurement device 2 may be a finger oximeter. The finger oximeter includes a clamp member (not shown in the drawings) used to clamp a finger of the user 5. The physiological signal interface 20 is arranged inside the clamp member and able to output the detection signal 21, such as an infrared ray. After the detection signal 21 is projected to the finger inside the clamp member, the measured physiological response signal 23, such as a signal including the blood oxygen signal, is transmitted to the blood oxygen measurement device 2 through the physiological signal interface 20 for analysis. The user 5 may learn the measurement result 25 via viewing the display module 24, such as a display screen of the finger oximeter. In addition to the blood oxygen level, the measurement results 25 may further, but not limit to, include blood pressure, pulse rate, atrial fibrillation (Af) or not, arrhythmia or not, or premature ventricular contractions or not. The blood oxygen measurement device 2 may be a wearable device able to be worn on the user for continuous long time. The signal processing-analyzing module 22 is connected with the transmission module 26 to transmit a portion or all of the measurement results 25. For example, a portion or all of the measurement results 25 are transmitted in a wired or wireless way. The signal processing-analyzing module 22 may include a processor, a memory and related circuits, but not limit to.

The transmission module 26 may include a wireless or wired transmitter to transmit wireless signals such as a Bluetooth signal, but not limit to.

Refer to FIG. 1 again. The oxygen supply device 4, for example but not limited to, an oxygen generator or an oxygen cylinder or aforementioned integrated together, at least includes an output interface 40 connected to a processing module 42. The processing module 42 may concentrate or supply sufficient amount of high-concentration oxygen, e.g. the concentration of oxygen more than 60% or better more than 90% with tolerances of +6% and −3%. The first flow rate of gas 41 containing high-concentration oxygen is output to the external of the oxygen supply device 4 through the output interface 40. In general application, the output interface 40 is directly connected with a gas output tube and a mask (not shown in the drawings). The first flow rate of gas 41 containing high-concentration oxygen may be directly provided to the nose and or mouth of the user 5 with the mask. In the present invention, an output flow of 1-5 liter per minute (first flow rate) may be executed by the oxygen supply device 4, and the output interface 40 of the oxygen supply device 4 is connected with the artificial intelligence oxygen controller 3. The artificial intelligence oxygen controller 3 at least includes an output module 30, a control module 32 and a gas regulation block 36. The control module 32 of the artificial intelligence oxygen controller 3 receives a physiological value 27 from the blood oxygen measurement device 2. The physiological values 27 at least include an instant blood oxygen level of the user 5. According to the physiological value 27, the control module 32 determines a second flow rate of gas 31 containing high-concentration oxygen which is smaller than or equal to the first flow rat of gas 41 containing high-concentration oxygen, for example, of 0%-100%. Via controlling the gas regulation block 36, the control module 32 regulates the amount of high-concentration oxygen that is finally taken by the user 5.

Refer to FIG. 1 again. In one embodiment, the control module 32 may stores a blood oxygen threshold range, such from 88 to 96. After comparing the blood oxygen level learned from the physiological values 27 with the blood oxygen threshold range, the control module 32 determines the second flow rate of gas 31 containing high-concentration oxygen to be formed by regulating the first flow rate of gas 41 containing high-concentration oxygen. It is understood that there may be different blood oxygen threshold range for specific patients, so the blood oxygen threshold range may be set by professional doctor and then stored for the control module 32, not to be limited to aforementioned. In one embodiment, the gas regulation block 36 includes a T-joint and a valve device arranged in the T-joint. The first channel of the T-joint is for first flow rate of gas 41 containing high-concentration oxygen flowing in, the second channel is an air channel and the third channel is for the second flow rate of gas 31 containing high-concentration oxygen. On condition that the measured blood oxygen level is smaller than the minimum of the blood oxygen threshold range (it indicates that the instant blood oxygen level of the user 5 is too low) after the comparison, the control module 32 turns off the air channel and lets the first flow rate of gas 41 containing high-concentration oxygen directly flow to the third channel for the second flow rate of gas 31 containing high-concentration oxygen, such that the second flow rate of gas 31 containing high-concentration oxygen is equal to the first flow rate of gas 41 containing high-concentration oxygen and outputted through the output module 30 to the user 5. On condition that the blood oxygen level is within the blood oxygen threshold range after the comparison, the control module 32 may open a portion of the air channel to form the second flow rate of gas 31 containing high-concentration oxygen by exhausting a portion of the first flow rate of gas 41 containing high-concentration oxygen from the air channel. Then, the second flow rate of gas 31 containing high-concentration oxygen is provided to the user 5 via the output module 30. Provided that the blood oxygen level is greater than the maximum of the blood oxygen threshold range after the comparision, the control module 32 may turn off a portion or all of the first flow rate of gas 41 containing high-concentration oxygen, whereby the second concentration of oxygen of the second flow rate of gas 31 may be zero or smaller than the first flow rate of gas 41 containing high-concentration oxygen. Besides, the artificial intelligence oxygen controller 3 may further include an input button module or an input interface for users to set the blood oxygen threshold range.

Refer to FIG. 1 again. The artificial intelligence oxygen control system 1 of the present invention receives the instant blood oxygen level of the user 5 from the blood oxygen measurement device 2 through the artificial intelligence oxygen controller 3, determines the instant high-concentration oxygen requirement of the user 5, and regulates the instant flow rate of gas containing the high-concentration oxygen supplied to the user 5. For example, if a homecare patient needing high oxygen supply uses the artificial intelligence oxygen control system 1 of the present invention, the oxygen supply may be regulated according to the instant blood oxygen level to satisfy the oxygen requirement of the patient without interrupting the sleep of the patient. Thus, the burden of the caregiver is also lessened. Since the flow rate of gas containing high-concentration oxygen supplied to the user is based on the user's instant oxygen level, the user is exempted from being compelled to continue inhaling high-concentration oxygen even though the blood oxygen of the user has been restored to the normal value. In practice, the blood oxygen measurement device 2 may be a finger oximeter or a wearable ring to clamp or wear the finger of the user 5 to measure the blood oxygen. The artificial intelligence oxygen controller 3 may be an independent device having a first connection port and a second connection port. The first connection port may be connected with the oxygen output port of the oxygen supply device 4. The second connection port may be connected with a hose and a respiration mask to supply oxygen to the user 5. The artificial intelligence oxygen controller 3 may further include an additional connection port, which may be connected with another gas supply device or function as an access hole of air. The artificial intelligence oxygen controller 3 of the present invention may match with the existing blood oxygen measurement device and oxygen supply device 4 to provide the user with a convenient and safe oxygen supply system.

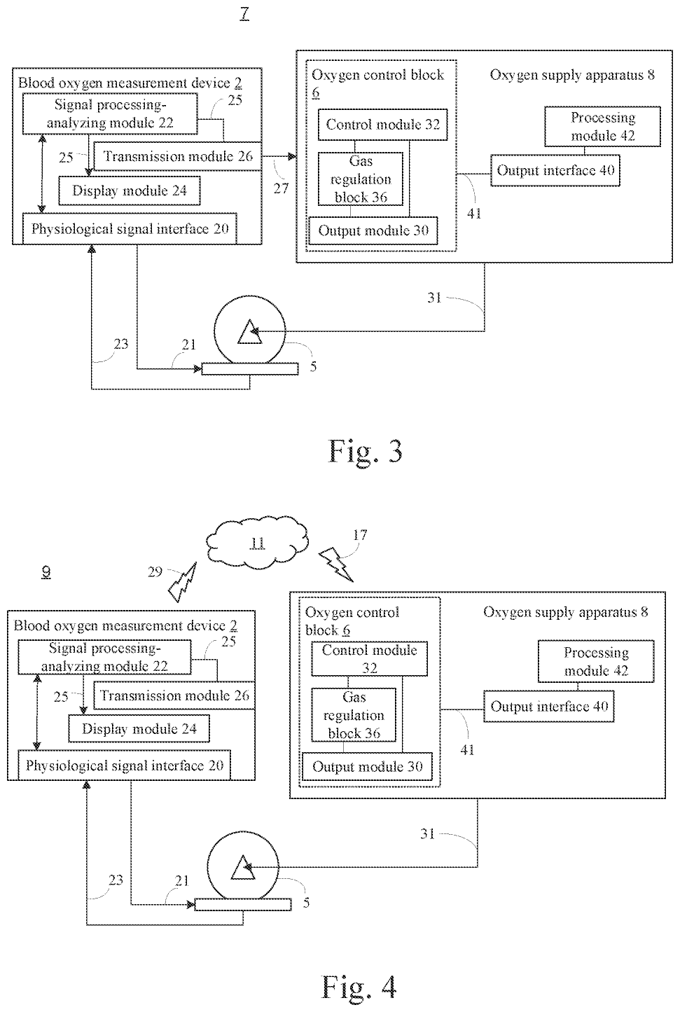

The artificial intelligence oxygen control system 1 of the present invention may have different embodiments. Refer to FIG. 2, which is a diagram schematically showing a artificial intelligence oxygen control system according to a second embodiment of the present invention. In comparison with FIG. 1, the artificial intelligence oxygen control system 5 in FIG. 2 comprises an artificial intelligence oxygen controller 3, wherein the gas regulation block 36 and the output module 30 of the artificial intelligence oxygen controller 3 are integrated into a gas-regulating output module 38. The gas-regulating output module 38, which may be implemented to all embodiments of the present invention, may include an oxygen input 35 for receiving the first flow rate of gas 41 and an oxygen output 37 for supplying the second flow rate of gas 31, such as channels and valve aforementioned. Refer to FIG. 3, which is a diagram schematically showing an artificial intelligence oxygen control system according to a third embodiment of the present invention. The artificial intelligence oxygen control system 7 in FIG. 3 includes a blood oxygen measurement device 2 and an oxygen supply apparatus 8, wherein the oxygen supply apparatus 8 includes an oxygen control block 6. The oxygen supply apparatus 8 in FIG. 3 is equivalent to the integration of the artificial intelligence oxygen controller 3 and the oxygen supply device 4 in FIG. 1. The oxygen control block 6 is equivalent to the artificial intelligence oxygen controller 3. It is easily understood: in the oxygen supply apparatus 8, the control module 32 and the processing module 42 may be integrated together, and the output module 30, the gas regulation block 36 and the output interface 40 may be integrated together. It is further explained: the integration of the control module 32 and the processing module 42 receives the physiological values 27 from the blood oxygen measurement device 2, acquires an instant blood oxygen level from the physiological values 27, compares the blood oxygen level with the blood oxygen threshold range, and outputs a blood oxygen control signal; the integration of the output module 30, the gas regulation block 36 and the output interface 40 receives the blood oxygen control signal to regulate the generation of oxygen, regulate the supply of air and output the second flow rate of gas 31 containing high-concentration oxygen.

Refer to FIG. 4, which is a diagram schematically showing a artificial intelligence oxygen control system according to a fourth embodiment of the present invention. The artificial intelligence oxygen control system 9 in FIG. 4 comprises a blood oxygen measurement device 2 and an oxygen supply apparatus 8, wherein a vital sign 29 of the blood oxygen measurement device 2 is transmitted to a cloud server 11 in a wireless way. In comparison with FIG. 3, the oxygen supply apparatus 8 in FIG. 4 can wirelessly communicate with the cloud server 11 to acquire physiological values 17 corresponding to the vital sign 29 of the blood oxygen measurement device 2, wherein the physiological values 17 at least include a blood oxygen level of a user 5. The other parts of the artificial intelligence oxygen control system 9 are the same as those of the artificial intelligence oxygen control system 7 and will not repeat herein. It is easily understood: the oxygen supply apparatus 8 may directly receive the physiological values (including the blood oxygen level) from the blood oxygen measurement device 2 in a wired or wireless way. The present invention does not limit that the physiological values must be transmitted in the way shown in FIG. 4. The vital sign 29 of the blood oxygen measurement device 2 may be periodically transmitted to the cloud server 11 for storage and then transmitted to another device persistently. Thereby, the user 5 can check the measurement results obtained by the blood oxygen measurement device 2 at specified time points or within a time interval. Thus, the user 5 can learn and administrate his own physiological information conveniently. Besides, the cloud server 11 may further present the measurement results on another display screen (not shown in the drawings). Furthermore, the measurement results 25, such as blood oxygen level, blood pressure, pulse rate, atrial fibrillation (Af) or not, arrhythmia or not, or premature ventricular contractions or not, may be transmitted in a wired or wireless way to the cloud server 11 for storing or utilizing.

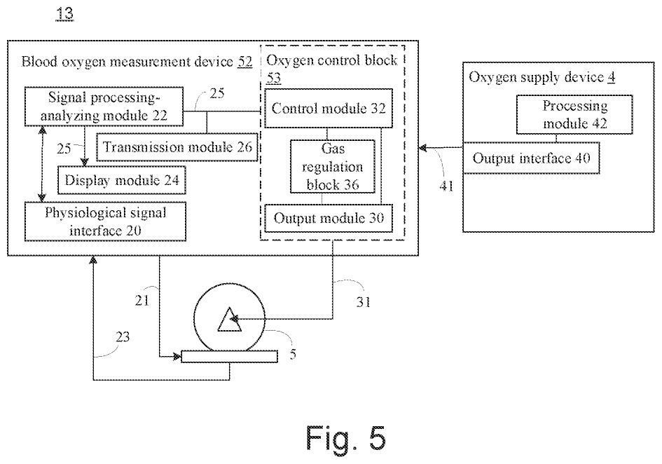

Refer to FIG. 5, which is a diagram schematically showing a artificial intelligence oxygen control system according to a fifth embodiment of the present invention. In comparison with FIG. 1, the artificial intelligence oxygen control system 13 in FIG. 5 comprises a blood oxygen measurement device 52. The blood oxygen measurement device 52 includes an oxygen control block 53. In other words, the artificial intelligence oxygen controller 3 and the blood oxygen measurement device 2 in FIG. 1 are integrated together in FIG. 5. In the fifth embodiment, the control module 32 of the oxygen control block 53 and the processing and analyzing module 22 may be integrated together, and the measurement result 25 includes a blood oxygen level. It is easily understood: the control signal 33 output by the blood oxygen measurement device 52 may be transmitted to the oxygen supply device 4 in a wired or wireless method. It is easily understood: the measurement result of the blood oxygen measurement device 52 may be sent out through the transmission module in a wired or wireless method. The details thereof will not repeat herein.

The embodiments described above are to demonstrate the technical thoughts and characteristics of the present invention to enable the persons skilled in the art to understand, make, and use the present invention. However, these embodiments are not intended to limit the scope of the present invention. Any equivalent modification or variation according to the spirit of the present invention is to be also included by the scope of the present invention.

What is claimed is:

1. An artificial intelligence oxygen controller, comprising:
an oxygen input receiving a first flow rate of gas containing high-concentration oxygen,
an oxygen output outputting a second flow rate of gas containing high-concentration oxygen, and
a gas regulation-output module receiving a blood oxygen level and comparing the blood oxygen level with a blood oxygen threshold range, where an amount of second flow rate of gas containing high-concentration oxygen from the first flow rate of gas containing high-concentration oxygen is determined by a comparison result.

2. The artificial intelligence oxygen controller according to claim 1, wherein the gas regulation-output module receives the blood oxygen level that is inputted in the artificial intelligence oxygen controller in a wireless or wired way.

3. The artificial intelligence oxygen controller according to claim 1, wherein the blood oxygen threshold range is stored in the gas regulation-output module.

4. An artificial intelligence oxygen control system including the artificial intelligence oxygen controller of claim 1, comprising:
a blood oxygen measurement device measuring a physiological response signal of a user, processing the physiological response signal to acquire the blood oxygen level and transmitting the blood oxygen level to the artificial intelligence oxygen controller; and
an oxygen supply device providing the first flow rate of gas containing high-concentration oxygen to the artificial intelligence oxygen controller, wherein a concentration of oxygen in the first flow rate of gas containing high-concentration oxygen is more than 60%.

5. The artificial intelligence oxygen control system according to claim 4, wherein the blood oxygen measurement device includes a signal processing-analyzing module connected with a physiological signal interface and a transmission module, wherein the physiological signal interface receives the physiological response signal from the user, the signal processing-analyzing module processes the physiological response signal to acquire a measurement result including the blood oxygen level, and the transmission module transmits the measurement result to a cloud server.

6. The artificial intelligence oxygen control system according to claim wherein the blood oxygen measurement device further include a display module displaying the measurement result.

7. The artificial intelligence oxygen control system according to claim 6, wherein the measurement result comprises blood oxygen level, blood pressure, pulse rate, atrial fibrillation (Af) or not, arrhythmia or not, or premature ventricular contractions or not or the combination of more aforementioned.

8. The artificial intelligence oxygen control system according to claim wherein the blood oxygen level is transmitted from the cloud server to the artificial intelligence oxygen controller.

9. The artificial intelligence oxygen control system according to claim 4, wherein while the blood oxygen level is smaller than a minimal value of the blood oxygen threshold range, the second flow rate of gas containing high-concentration oxygen is equal to the first flow rate of gas containing high-concentration oxygen; and while the blood oxygen level is larger than a maximum value of the blood oxygen threshold range, the second flow rate of gas containing high-concentration oxygen is zero or smaller than the first flow rate of gas containing high-concentration oxygen.

10. The artificial intelligence oxygen control system according to claim 4, wherein the blood oxygen measurement device is a finger-clamping blood oxygen device or a wearable type blood oxygen device.

11. The artificial intelligence oxygen control system according to claim 4, wherein oxygen supply device is an oxygen generator or an oxygen cylinder.

12. The artificial intelligence oxygen control system according to claim 4, wherein the blood oxygen threshold range is stored in the gas regulation-output module.

13. A blood oxygen measurement device including the artificial intelligence oxygen controller of claim 1, comprising
a physiological signal interface outputting a detection signal and receiving a physiological response signal of the user, wherein the detection signal interferes with the user to generate the physiological response signal of the user;
a signal processing-analyzing module connected with the physiological signal interface and processing the physiological response signal to acquire a measurement result including the blood oxygen level;
a transmission module connected with signal processing-analyzing module, transmitting the measurement result to an external device for presentation in a wireless or wired way; and
a display module connected with the signal processing-analyzing module for displaying the measurement result.

14. The blood oxygen measurement device according to claim 13, wherein while the blood oxygen level is smaller than a minimal value of the blood oxygen threshold range, the second flow rate of gas containing high-concentration oxygen is equal to the first flow rate of gas containing high-concentration oxygen; and while the blood oxygen level is larger than a maximum value of the blood oxygen threshold range, the second flow rate of gas containing high-concentration oxygen is zero or smaller than the first flow rate of gas containing high-concentration oxygen.

15. The blood oxygen measurement device according to claim 13, wherein the blood oxygen threshold range is stored in the gas regulation-output module.

16. An artificial intelligence oxygen supply device including the artificial intelligence oxygen controller of claim 1, comprising an oxygen supplier providing the first flow rate of gas containing high-concentration oxygen to the artificial intelligence oxygen controller, wherein a concentration of oxygen in the first flow rate of gas containing high-concentration oxygen is more than 60%.

17. The blood oxygen measurement device according to claim 16, wherein the gas regulation-output module receives the blood oxygen level that is inputted in the artificial intelligence oxygen controller in a wireless or wired way.

18. The blood oxygen measurement device according to claim 16, wherein the blood oxygen threshold range is stored in the gas regulation-output module.

19. The blood oxygen measurement device according to claim 16, wherein while the blood oxygen level is smaller than a minimal value of the blood oxygen threshold range, the second flow rate of gas containing high-concentration oxygen is equal to the first flow rate of gas containing high-concentration oxygen; and while the blood oxygen level is larger than a maximum value of the blood oxygen threshold range, the second flow rate of gas containing high-concentration oxygen is zero or smaller than the first flow rate of gas containing high-concentration oxygen.
